(19) United States
(12) Patent Application Publication
(10) Pub. No.: US 2007/0207225 A1
(43) Pub. Date: Sep. 6, 2007

(54) GENISTEIN MODULATED REDUCTION OF CARDIOVASCULAR RISK FACTORS

(76) Inventor: FRANCESCO SQUADRITO, Messina (IT)

Correspondence Address: NOBLITT & GILMORE, LLC., 4800 NORTH SCOTTSDALE ROAD, SUITE 6000, SCOTTSDALE, AZ, 85251, US

(21) Appl. No.: 11/682,042

(22) Filed: Mar. 5, 2007

Related U.S. Application Data
(60) Provisional application No. 60/779,260, filed on Mar. 3, 2006.

(51) Int. Cl. A61K 31/353; A61K 36/48

(52) U.S. Cl. 424/757; 514/456

(57) ABSTRACT

The disclosed methods and compositions for reducing cardiovascular risk factors in mammals generally includes using genistein to modulate various inflammatory and cardiovascular risk markers including: homocysteine, C-reactive protein, fibrinogen, sex hormone-binding globulin, fasting glucose, insulin, insulin resistance, and osteoprotegerin.

### FIELD OF INVENTION

The present invention generally concerns cardiovascular function, and more particularly, representative and exemplary embodiments of the present invention generally relate to reduction of cardiovascular risk factors with the administration of soy isoflavones to affect inflammatory and cardiovascular markers in mammals.

### BACKGROUND OF INVENTION

The importance of the soybean plant in Eastern civilizations predates written records. As a legume, the soybean plant, Glycine max, has historically been used for food and fuel in Asia. It has only been in the last few centuries that Western cultures have recognized and utilized the soy bean plant. Only even more recently have some of the nutritional benefits of soybean been studied.

Epidemiological evidence in Asians shows a link between consumption of soy and decreases in post-menopausal symptoms, osteoporotic related fractures, and certain neoplasia. Additionally, studies in rabbits and humans have demonstrated that soy protein rich diets result in lower cholesterol levels.

While numerous studies have established the benefits of soy protein, only a few studies concerning the benefits of isoflavone extracts (e.g., phytoestrogens) have been performed with incomplete and/or contradictory results. For example, isoflavone extracts have been previously shown to have no appreciable effect on lipid levels in a meta-analysis of human clinical trial data. More recently, isoflavone mixtures were shown to have no effect on lipid, glucose or insulin levels. Moreover, comparative trials have demonstrated greater effects on lipid levels by soy protein and soy protein extracts as compared with soy isoflavone extracts. Furthermore, the majority of soy isoflavone research has been carried out on poorly characterize mixtures containing a substantial compositional fraction of impurities. For example, it has been previously demonstrated that a phytoestrogen-rich soy protein diet reduces LDL and very low density (vLDL) cholesterol concentrations in primates.

It is desirable to have a deeper understanding of the interaction between hormone-related changes and metabolic changes in lipids, insulin, body fat distribution, and the like for post-menopausal women. It is recognized that there is an increase in incidence of diseases, like coronary artery disease, after menopause in females; however, hormone replacement therapy has not been shown to be effective in reducing the incidence of cardiovascular events. This and other adverse effects of hormone replacement therapy, such as increased incidence of breast cancer, endometrial cancer, and thromboembolic events, have inspired recognition of the need for alternatives to conventional hormone therapy.

### SUMMARY OF THE INVENTION

In various representative aspects, the present invention provides a method for reducing cardiovascular risk factors in mammals using genistein. Reduction of risk factors may occur through modulating (i.e., increasing and/or decreasing) various inflammatory and cardiovascular risk markers including: homocysteine, C-reactive protein, fibrinogen, sex hormone-binding globulin, fasting glucose, fasting insulin, insulin resistance, and osteoprotegerin.

Advantages of the present invention will be set forth in the Detailed Description which follows and may be apparent from the Detailed Description or may be learned by practice of exemplary embodiments of the invention. Still other advantages of the invention may be realized by means of any of the instrumentalities, methods or combinations particularly pointed out in the claims.

### BRIEF DESCRIPTION OF THE DRAWINGS

Representative elements, operational features, applications and/or advantages of the present invention reside inter alia in the details of construction and operation as more fully hereafter depicted, described and claimed—reference being made to the accompanying drawings forming a part hereof, wherein like numerals refer to like parts throughout. Other elements, operational features, applications and/or advantages will become apparent in light of certain exemplary embodiments recited in the detailed description, wherein:

FIG. 1 representatively illustrates the chemical structure of genistein;

FIG. 2 representatively illustrates levels of C-reactive protein of osteopenic women treated with pure genistein versus placebo and hormone replacement therapy;

FIG. 3 representatively illustrates levels of homocysteine protein of osteopenic women treated with pure genistein versus placebo and hormone replacement therapy;

FIG. 4A representatively illustrates levels of estradiol in osteopenic women treated with placebo and genistein (represented by the hatched markings) at 0 months and 6 months;

FIG. 4B representatively illustrates levels of genistein in osteopenic women treated with placebo and genistein (represented by the hatched markings) at 0 months to 6 months;

FIG. 5 representatively illustrates levels of fasting glucose in osteopenic women treated with placebo and genistein (represented by the hatched markings) at 0 months and 6 months:

FIG. 6 representatively illustrates levels of insulin in osteopenic women treated with placebo and genistein (represented by the hatched markings) at 0 months and 6 months;

FIG. 7 representatively illustrates levels of insulin resistance in osteopenic women treated with placebo and genistein (represented by the hatched markings) at 0 months and 6 months, and

FIG. 8 representatively illustrates levels of osteoprotegerin in osteopenic women treated with placebo and genistein (represented by the hatched markings) at 0 months and 6 months.

Elements in the Figures are illustrated for simplicity and clarity and have not necessarily been drawn to scale. For example, the dimensions of some of the elements in the Figures may be exaggerated relative to other elements to help improve understanding of various embodiments of the present invention. Furthermore, the terms “first”, “second”, and the like herein, if any, are used inter alia for distinguishing between similar elements and not necessarily for describing a sequential or chronological order.

### DETAILED DESCRIPTION OF EXEMPLARY EMBODIMENTS

The following representative descriptions of the present invention generally relate to exemplary embodiments and the inventor's conception of the best mode, and are not intended to limit the scope, applicability or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Various representative implementations of the present invention may be applied to any system for reduction of cardiovascular risk factors using genistein. Certain representative implementations may include, for example, the administration of: soy, soy extracts, soy isoflavone extracts, hormones, calcium, vitamins, minerals and/or the like.

As used herein, the terms “treatment” or “treated”, or any contextual variant thereof, are generally intended to describe any administration of a pharmaceutically active dose of an active agent to achieve biological change and/or the like.

As used herein, the term “therapeutic” or “therapy”, or any contextual variant thereof, are generally intended to describe treatment and/or prophylaxis in mammals and the like.

As used herein, the terms “pharmaceutically effective dose”, “pharmaceutically effective amount”, “therapeutically effective dose”, “therapeutically effective amount”, or any contextual variant thereof are generally intended to describe any dosage level sufficient to induce a desired biological effect.

As used herein, the term “placebo”, or any contextual variant thereof, is generally intended to describe the substitution of a pharmaceutically or therapeutically effective dose or amount sufficient to induce a desired biological change with a non-active substance.

As used herein, the term “patient” or “individual”, or any contextual variant thereof, are generally intended to describe a living subject—human or animal.

A detailed description of an exemplary application, namely a method for improving cardiovascular function, is provided as a specific enabling disclosure that may be generalized to any application of the disclosed system, composition and method for reducing cardiovascular risk factors in mammals in accordance with various representative embodiments of the present invention.

The present invention relates to the use of particular soy isoflavones known as phytoestrogenic genistein or genisteol. Genistein may be found in a number of isoflavone containing plants or isolated from Glycine max.

Isoflavones are present in food as glycosides and malonates, which are hydrolyzed in the gut by the intestinal flora and mucosal cells. See Q. Wu, M. Wang, W. J. Sciarappa and J. E. Simon, “LC/UV/ESI-MS Analysis of Isoflavones in Edamame and Tofu Soybeans”, J. Agric. Food Chem. 52 (2004), pp. 2763-2769; K. D. Setchell, N. M. Brown, L. Zimmer-Nechemias, W. T. Brashear, B. E. Wolfe, A. S. Kirschner and J. E. Heubi, “Evidence for Lack of Absorption of Soy Isoflavone Glycosides in Humans, Supporting the Crucial Role of Intestinal Metabolism for Bioavailability”, Am. J. Clin. Nutr. 76 (2002), pp. 447-453; K. D. Setchell, N. M. Brown, P. Desai, L. Zimmer-Nechemias, B. E. Wolfe, W. T. Brashear, A. S. Kirschner, A. Cassidy and J. E. Heubi, “Bioavailability of Pure Isoflavones in Healthy Humans and Analysis of Commercial Soy Isoflavone Supplements”, J. Nutr. 131 (2001), pp. 1362S-1375S. In competition assays, glycosides of genistein and diadzein showed only weak binding to estrogen receptors ERα and ERβ, whereas the aglycone forms bound avidly to both receptors. Genistein's affinity to bind to classical estrogen receptor is higher than the binding of other isoflavones, and in addition, the binding is stronger to ERβ than to ERα. Metabolites of these substances, such as equol and dihydrogenistein, show binding similar to the isoflavones, but lower than genistein. See K. Morito, T. Hirose, J. Kinjo, T. Hirakawa, M. Okawa, T. Nohara, S. Ogawa, S. Inoue, M. Muramatsu and Y. Masamune, “Interaction of Phytoestrogens with Estrogen Receptors Alpha and Beta”, Biol. Pharm. Bull. 24 (2001), pp. 351-356. Genistein's ability to bind to ERα receptors allows genistein to act as a selective estrogen receptor modulator with both agonist (binding ER-β) and antagonist (binding ER-α) activity, and thereby compete with endogenous. Moreover, in addition to competing with endogenous estrogens for binding to the estrogen receptor, genistein may exert anti-estrogenic effects by several potential mechanisms, including, but not limited to, modulating inflammatory and/or cardiovascular risk markers.

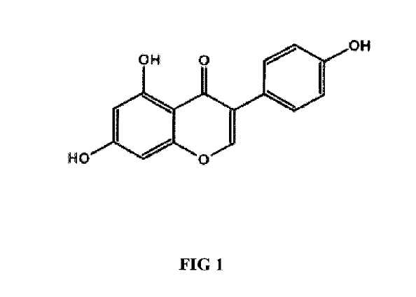

In soy and soy products, 95-99% of genistein exists in the conjugated form genistein (glycoside). Referring now to FIG. 1, the unconjugated form of genistein (aglycone), with the IUPAC nomenclature: 4′,5,7-Trihydroxyisoflavone 5,7-Dihydroxy-3-(4-hydroxyphenyl)-4H-1-benzopyran-4-one, occupies only 1-5% in soy or soy-derived products. Thus, people having a relatively high soy protein diet are regularly exposed to genistin (glycoside) far more than genistein (aglycone) when consuming soy and soy-derived products in their diet. However, when these foods containing mostly genistin are processed by O-glucosideases in the intestinal brush boarder and bacterial flora in the gastrointestinal tract, the metabolic by-products of genistin isoflavones may produce different effects on the body.

Genistein, according to various aspects of the present invention, may comprise the aglycone form. In one representative embodiment, the aglycone form of genistein, also known as “pure” or “substantially pure” genistein, may be administered as a composition to a patient in a pharmaceutically active dose. By providing the aglycone form of genistein, according to the present invention, the potential for agonistic effects on the body may be minimized, and efficiency of uptake may be increased.

The aglycone form of genistein, according to various aspects of the present invention, may be produced by a proprietary process of soy fermentation, extraction and precipitation. In accordance with this processing method, genistein may be obtained in concentrations of >98% (wt/wt) as compared to pure genistein standards by HPLC-MS.

Genistein, according to various aspects of the present invention, may be suitably adapted for co-administration with a pharmaceutically active dose of calcium, vitamin D3, zinc, and/or the like. “Genistein compositions”, as hereinafter described, may comprise genistein, and/or genistein in combination with calcium, vitamin D3, and/or zinc and/or the like. Calcium, vitamin D3, and zinc, according to various aspects of the present invention, may comprise any dose and may be derived from any variety of sources, including synthetic or natural sources.

In a representative embodiment of the present invention, genistein compositions may be administered orally, with a weight of approximately 54 mg/d, in a bolus, in a metered fashion, in a time-release fashion, and/or daily. In another representative embodiment of the present invention, genistein compositions may be suitably configured to comprise a pharmaceutically active composition suitable for achieving a biological effect, including but not limited to maintaining, normalizing, increasing and/or reducing inflammatory and/or cardiovascular markers, decreasing the risk of a chronic disease, decreasing the likelihood of worsening an existing chronic disease and/or the like. In yet a further exemplary embodiment of the present invention genistein compositions may be suitably configure to: reduce negative side effect of hormone replacement therapy; enhance dilator response to acetychlioline of atheroscelerotic arteries; reduce risk of at least one of coronary heart disease, venous thrombolism, and metabolic hepatic activation; improve endothelial dependent vasodilation; comprise at least one of an anti-neoplastic effect and an anti-mutagenic effect, and at least partially inhibit LDL oxidation, endothelial cell proliferation, and/or angiogenesis.

Genistein compositions, according to various representative embodiments of the present invention, may be suitably administered to regulate, stabilize and/or decrease levels of inflammatory markers, where such inflammatory markers may comprise any marker that at least partially leads to observance of an increased risk of a chronic disease or at least partially leads to a worsening of an existing chronic disease. Inflammation is part of the etiology of cardiovascular disease. In one representative embodiment, genistein compositions may be utilized as a therapeutic treatment of cardiovascular disease. In another representative embodiment, genistein compositions may be administered to reduce the risk of worsening cardiovascular disease as a consequence of other treatments for other conditions.

Inflammatory markers, according to various representative embodiments of the present invention, may comprise G-reactive proteins and/or homocysteine and/or the like. Table 1 representatively illustrates the differences in inflammatory markers (plasma homocysteine (μmol/l) and C-reactive proteins levels (CRP, mg/l)) in osteopenic women treated with genistein (n=30, 54 mg/day) in accordance with exemplary embodiments of the present invention versus hormone replacement therapy (HRT) (n=30; estradiol 1 my and norethisterone acetate 0.5 mg), and placebo (n=30) during a six month period. Prior to the six month period, participants in the study were placed on a standard fat-reduced diet for four-weeks, which constituted a stabilization process. The participants were then randomly assigned to receive either genistein, HRT or a placebo. Throughout the study period, samples were obtained while participants were fasting to minimize dietary effects. The samples were used to measure serum C-reactive protein and plasma homocysteine.

Serum C-reactive protein was measured using an immunochemiluminescent assay (Immulite DPC, Los Angeles, Calif., USA). Samples were stored after separation at −80° C. until assayed. Sensitivity was 0.07 μg/ml with intra- and interassay coefficient of variations of 3.1 and 5.7%, respectively. The reference interval was 0.20-5.10 μg/ml.

Plasma homocysteine was measured using an immunochemiluminescent assay (Immulite DPC, Los Angeles, Calif., USA). Samples were stored after separation at −80° C. until assayed. Homocysteine was assayed on plasma samples after treatment with S-adenosyl-L-homocysteine (SAH) hydrolase and dithiothreitol (DTT) solution. EDTA plasma was separated from the cells as soon as possible after collection. Samples were stored on ice between the time of sampling and centrifugation. The assay sensitivity was 0.8 μmol/l, the interassay coefficient of variation was 5.4% at 10:5 μmol/l and the reference interval was 5-17 μmol/l.

For statistical evaluation of the serum C-reactive protein and homocysteine, a two-way ANOVA with Bonferroni post-test was performed. See D'Anna R., Baviera B., Corrado F., Cancellieri F., Crisafulli A. and Squadrito F., et al., “The Effect of the Phytoestrogen Genistein and Hormone Replacement Therapy on Homocysteine and C-Reactive Protein Level in Postmenopausal Women”, Acta OBstst Gynecol Scand, 2005, 84: 474-477. The results in this table demonstrate that genistein, when administered, does not increase or fluctuate homocysteine and/or C-reactive protein levels,

TABLE 1
Table I. Plasma homocysteine (μmol/l) and serum C-reactive protein (CRP, mg/l) level in the genistein, HRT and placebo group

| | Placebo | | | Genistein | | | HRT | | |
|---|---|---|---|---|---|---|---|---|---|
| | Before (n = 30) | After (n = 28) | P | Before (n = 30) | After (n = 27) | P | Before (n = 30) | After (n = 26) | P |
| Homocysteine | 11.26 ± 0.33 | 11.5 ± 0.43 | ns | 11.36 ± 0.39 | 10.72 ± 0.46 | ns | 11.21 ± 0.44 | 10.45 ± 0.38 | ns |
| CRP | 1.69 ± 0.21 | 1.74 ± 0.22 | ns | 1.73 ± 0.31 | 2.13 ± 0.45 | ns | 1.61 ± 0.25 | 3.30 ± 0.55 | <0.01 |

Values represent mean ± SE. ns, non-signigicant

C-reactive proteins function as surrogate markers of inflammatory status in healthy as well as diseased individuals. Concentrations of C-reactive proteins in the blood can fluctuate widely in response to acute tissue damage, infection and/or the like. Recently, long-term, persistent, systematic inflammation or low-grade inflammation have been studied in individuals using C-reactive protein as a marker compared to levels of more extensively studied risk factors, such as blood cholesterol concentrations and blood pressure. See Danesh J., Collins R., Appleby P., Peto, R., 1998, JAMA, 279:1477; and Ridker P. M., Rifai N., Pfeffer M. A., Sacks F. M., Braunwald E., 1999, Circulation, 100:230. These studies have shown that increased risks of cardiovascular disease may be predicted through levels of C-reactive protein, but a causal link remains elusive. Even slightly elevated levels of C-reactive protein have been associated with a persistent low-grade inflammation due to arthrogenic leisures which can result in long-term damage to the cardiovascular system. Accordingly, increasing C-reactive protein levels by administration of a pharmaceutical preparation (as in hormone replacement therapy) or with the consumption of certain food substances may result in a greater risk of cardiovascular disease.

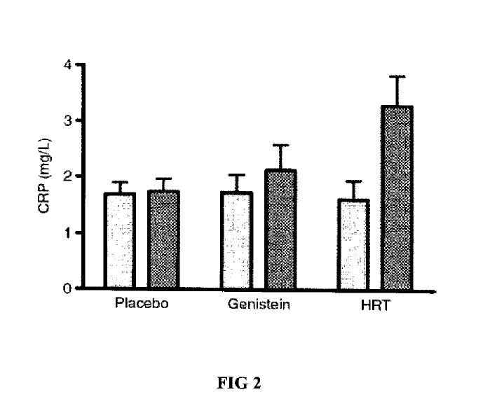

Referring now to FIG. 2, and as generally disclosed in the Table 1 above, C-reactive protein levels were relatively unchanged in post menopausal women during the aforementioned six month study with the administration of genistein. Specifically, there were no significant differences at the end of treatment from baseline in C-reactive protein levels in those women who were administered genistein. Additionally, no significant differences in C-reactive protein levels were present in those women who received placebo. By contrast, the mean C-reactive protein level was about two times higher than the baseline among women taking HRT for 6 months and this difference was statistically significant (P<0-01). Thus, while HRT increased C-reactive protein serum levels two-old, genistein did not significantly affect C-reactive protein serum levels.

It will be appreciated that genistein compositions, according to various aspects of the present invention, generally provide stability and/or regulate C-reactive proteins as inflammatory markers, and thereby may have the ability to decrease the risk of cardiovascular disease, venous thrombolism and/or the like.

In another representative embodiment, inflammatory markers in accordance with the present invention may comprise homocysteine. As a homologue of cysteine, a non-essential but important amino acid, homocysteine has an identical chemical formula to cysteine with the exception of an additional methylene group. Cysteine plays an important role in the body by cross-linking proteins.

In healthy, well-nourished individuals homocysteine metabolism is well regulated and the plasma concentration is usually less than 12 μM. Elevated levels of homocysteine, known as hyperhomocysteinemia, has been implicated as a risk factor for cardiovascular disease and is associated with various other diseases including neural tube defects, Alzheimer's disease, schizophrenia, acute renal disease, osteoporosis, and Type I diabetes and/or the like. See E. Eikelboom, J. W., Lonn E., Genest J. Jr., Hankey G., Yusuf S., 1999, Ann Intern Med. 131:363; Mocully K. S., 1969, Am J Pathology, 56:111, Clark R., Smith A. D., Jobst K. A., Refsum H., Sutton L., Ueland P. M., 1998, Arch Neurol. 55:1449; Mills J. L., McPartlin J. M., Kirke P. N., Lee Y. J., Conley M. R., Weir D. G., Scott J. M., 1995, Lancet 345:149; Applebaum J., Shimon H., Sela B. A., Belmaker R. H., Levine J, 2004, J Psychiatr Res 38:413; Van G. C., Stehouwer C. D., 2003, Clin Chem Lab Med 41:1412; Villadsen M. M., Bunger M. H., Carstens M., Stenkjaer L., Langdahl B. L., 2005 Osteoporos Int 16:411; Villadsen M. M., Bunger M. H., Carstens M., Stenkjaer L., Langdahl B. L., 2005, Osteoporos Int. 16:411; De Luis D. A., Fernandez N., Arranz M. L., Aller R., Izaola O., Romero E., 2005, J Diabetes Compl. 19:42; Rudy A., Kowalska I., Straczkowski M., Kinalska I., 2005, Diabetes Metab 31:112.

Hyperhomocysteinemia is often the result of genetic defects and/or nutritional deficiencies. While the mechanism(s) by which hyperhomocysteinemia causes diseases have not been fully elucidated, homocysteine is known to have the ability to modulate expression of certain genes that may either directly or indirectly lead to several pathological conditions. See Sharma P., Senthilkumar R., Brahmachari V., Sundaramoorthy E., Mahajan A., Sharma A., Sengupta S., 2006, “Mining Literature for a Comprehensive Pathway Analysis: A Case Study for Retrieval of Homocysteine Related Genes for Genetic and Epigenetic Studies”, Lipids Health Dis. 5:1. Accordingly, increasing homocysteine levels through administration of a pharmaceutical preparation or food substance could result in greater risk of diseases, including but not limited to cardiovascular disease, neural tube defects, Alzheimer's disease, schizophrenia, acute renal disease, osteoporosis, Type I diabetes and/or the like.

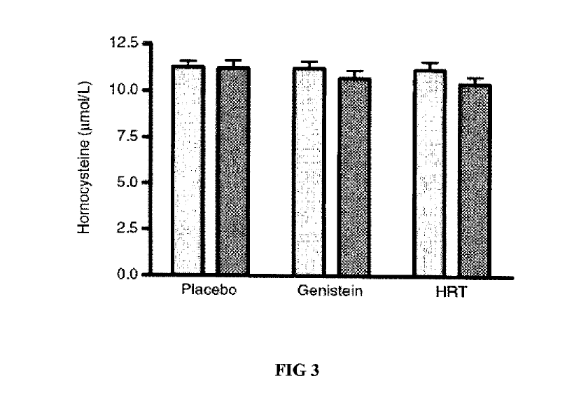

Referring now to FIG. 3, and as generally illustrated in Table 1 above, homocysteine levels were relatively unchanged with the administration of genistein. Specifically, the plasma homocysteine mean value was slightly decreased from baseline in the genistein and HRT group, but the difference was not statistically significant (P>0.05). Additionally, no significant difference in C-reactive protein levels was present in those women who received placebo (P>0.05).

It will therefore be appreciated that genistein, according to various aspects of the present invention, when administered, maintains, normalizes, and/or does not significantly affect homocysteine levels. Thus, genistein may not increase the risk linked to elevated circulating levels of homocysteine. Accordingly, genistein may decrease a patient's risk of acquiring, or alternatively (or conjunctively), worsening the condition of a variety of diseases, including but not limited to cardiovascular disease, neural tube defects, Alzheimer's disease, schizophrenia, acute renal disease, osteoporosis, Type I diabetes and/or the like.

Genistein compositions, according to various representative embodiments of the present invention, may be suitably administered to modulate levels of cardiovascular risk markers, where such cardiovascular risk markers may comprise any markers that at least partially leads to an increased risk of a chronic disease, or at least partially leads to a worsening of an existing chronic disease. In one representative embodiment, a composition comprising genistein compositions may be utilized as a therapeutic treatment for cardiovascular disease. In another representative embodiment, genistein compositions may be administered to reduce the risk of worsening cardiovascular disease as a consequence of other treatments for other conditions.

Cardiovascular risk markers, according to various embodiments of the present invention, may comprise fasting insulin levels, fasting glucose levels, insulin resistance levels, osteoprotegerin levels, sex hormone-binding globulin levels, fibrinogen levels, estradiol levels and/or the like. Table 2, below presents baseline levels of fasting insulin levels, insulin resistance levels, osteoprotegerin levels, sex hormone-binding globulin levels, fibrinogen levels, plasma genistein and estradiol levels in postmenopausal women prior to a six month period of treatment. According to Table 3, below, the differences in fasting insulin levels, insulin resistance levels, osteoprotegerin levels, sex hormone-binding globulin levels, fibrinogen levels, plasma genistein and estradiol levels in postmenopausal women who where administered genistein (n=30) and those who were administered a placebo (n=30) after a sixth month period of treatment is shown. See Crisafulli A., Altavilla D., Marini H., Bitto A., Cucinotta D., Frisina N., Corrado F., D'Anna R., Squadrito G., Adamo E., Marini R., Romeo A., Cancellieri F., Buemi M., Squadrito F., Menopause, The Journal of the North American Menopause Society, Vol. 12 No. 2 pp. 186-192.

Prior to this six month period, participants in the study were placed on a standard fat-reduced diet for four-weeks, which constituted a stabilization process. The participants were then randomly assigned to receive the phytoestrogen genistein (n=30; 54 mg/d, Lab Plants Messina, Italy) or placebo (n=30) for six months.

To measure estradiol and plasma genistein levels, serum glucose was measured by an enzymatic kit (BioSystem S. A., Barcelona, Spain), (intra-assay CV 1%; interassay CV 1.8%; lower detection limit, 0.0126 mmol/l). To evaluate estradiol and genistein plasma levels, blood samples (0.5 ml) were collected in polypropylene tubes containing 50 ml of heparin (50,000 IU) and after centrifugation at 3,000 g at 4° C. for 10 minutes, each sample was stored at −70° C. until analysis. The assay was performed by using an HPLC method with UV detection. The concentration of plasma genistein was expressed in μmol/l.

To measure fasting glucose levels, serum glucose was measured by an enzymatic kit (BioSystemSA, Barcelona, Spain), (intra-assay CV 1%; interassay CV 1.8%; lower detection limit, 0.0126 mmol/L). Glucose in the sample produces, by means of the coupled reactions, a colored complex that can be spectrophotometrically measured.

To measure fasting insulin levels, insulin was measured by a commercially available ELISA kit according to the protocol of the manufacturer (DRG Diagnostik, Frauenberg Germany) (intraassay CV 4%; interassay CV 6%; lower detection limit, <1.5 μlU/ml).

To measure insulin resistance levels, insulin was measured by a commercially available ELISA kit according to the protocol of the manufacturer (DRG Diagnostik, Frauenberg Germany) (intraassay CV 4%; interassay CV 6%; lower detection limit, <1.5 μlU/ml). Serum glucose was measured by an enzymatic kit (BioSystemSA, Barcelona, Spain), (intra-assay CV 1%; interassay CV 1.8%; lower detection limit, 0.0126 mmol/l). Glucose in the sample produces, by means of the coupled reactions, a colored complex that can be spectrophotometrically measured. Insulin resistance was calculated using the Homeostasis Model Assessment method (HOMA-IR=(insulin×glucose)/22.5).

To measure osteoprotegerin levels, a commercially available ELISA kit according to the protocol of the manufacturer (Immunodiagnostik Bensheim Germany) was utilized. This assay detects monomeric, dimeric, and ligand-bound forms of OPG (intra-assay CV 5%; interassay CV 6%; lower detection limit, 0.14 pmol/l).

To measure fibrinogen levels, automated routine procedures were utilized.

To measure sex hormone-binding globulin levels, an immunoradiometric assay (RADIM SPA, Rome, Italy) (intra-assay CV 4%; interassay CV 5%; lower detection limit, 2.5 nmol/L) was used.

Data was presented as mean±SEM, and the significance of the difference was assessed by analysis of variance, where a value of P less than 0.05 was considered statistically significant.

TABLE 2

| | Placebo (n = 30) | Genistein (n = 30) |
|---|---|---|
| Fasting Glucose | 5 ± 0.12 | 4.74 ± 0.11 |
| Fasting Insulin | 6.6 ± 0.85 | 7 ± 0.55 |
| Insulin resistance | 1.45 ± 0.20 | 1.47 ± 0.12 |
| Sex Hormone Binding Globulin | 75 ± 2.92 | 71 ± 4.2 |
| Fibrinogen | 3.7 ± 0.05 | 3.6 ± 0.12 |
| Osteoprotegerin | 4.98 ± 0.16 | 4.67 ± 0.13 |
| Estradiol | 71 ± 2.37 | 73 ± 2.19 |
| Plasma Genistein | 0.06 ± 0.002 | 0.07 ± 0.004 |

TABLE 3

| | Placebo (n = 30) | Genistein (n = 30) |
|---|---|---|
| Fasting Glucose | 5.3 ± 0.19 | 4.3 ± 0.10 |
| Fasting Insulin | 8.23 ± 0.71 | 6.24 ± 0.45 |
| Insulin resistance | 2 ± 0.21 | 1.18 ± 0.08 |
| Sex Hormone Binding Globulin | 53 ± 2.92 | 63 ± 3.83 |
| Fibrinogen | 3.83 ± 0.04 | 3.18 ± 0.12 |
| Osteoprotegerin | 5.5 ± 0.13 | 4.4 ± 0.11 |

The results of Table 3 generally indicate that administration of genistein: increases plasma genistein without significantly affecting estradiol levels, reduces fasting glucose levels, reduces fasting insulin levels, reduces insulin resistance levels, reduces osteoprotegerin levels, reduces fibrinogen levels, and reduces sex hormone-binding globulin levels.

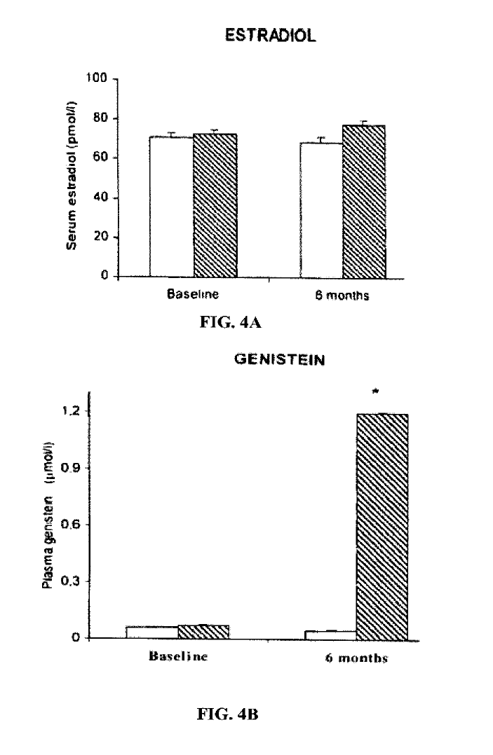

Referring now to FIG. 4, in one representative embodiment of the present invention, cardiovascular risk markers according to the present invention may comprise estradiol. FIG. 4A illustrates that genistein, according to various embodiments of the present invention, given over a six month period does not significantly elevate estradiol levels. Specifically, the baseline levels of those women administered genistein was approximately 74 pmol/L, and the six month readings were approximately 78 pmol/L. The baseline levels in those women given placebo was approximately 70 pmol/L, and after 6 months the estradiol levels were slightly decreased to approximately 68 pmol/L. FIG. 4B illustrates that genistein levels attain a high stead-state compared to placebo. Specifically, baseline levels of plasma genistein in those women administered genistein were approximately 0.75 μmol/L, and after 6 months, the plasma genistein levels were elevated to approximately 1.2 μmol/L. This increase in plasma genistein levels after 6 months of treatment with genistein is approximately 60%. By comparison, the plasma genistein levels in women who were administered placebo slightly decreased from approximately 0.72 μmol/L to approximately 0.68 μmol/L, a 5.6% decrease.

Thus, genistein does not stimulate estradiol levels as genistein plasma levels are elevated. By not affecting the estradiol levels, genistein may provide an alternative to HRT for treating chronic diseases, like cardiovascular disease, without increasing the risk of cancers, such as breast cancer and ovarian cancer.

In one representative embodiment of the present invention, the cardiovascular risk marker according to the present invention comprises fasting glucose. Glucose is a monosaccharide, a simple six carbon sugar, and is a main source of energy in humans. In healthy individuals, glucose concentrations are tightly regulated through a balance between glucose uptake from the blood and deposition of glucose into the liver and other tissues. See Clutter W. E., Cryer P. E. 1990, Hypoglycemia Stein J. H., ed. Internal Medicine, Boston, Mass., Little Brown & Co, pgs. 2267-2272. Fasting glucose levels which are lower lead to a decreased risk of diabetes and/or decreased risk in elevating the condition of diabetes. Moreover, fasting glucose levels and diabetes are positively associated with the increase incidence of cardiovascular disease as well as the incidence and mortalities from other disease states. See Geiss L. S., Herman W. H., Smith P. J., 1995, “Mortality in Non-Insulin-Dependent Diabetes”, National Diabetes Data Group, ed., Diabetes in America, Bethesda, Md., National Institutes of Health, National Institute of Diabetes and Digestive and Kidney Diseases, pgs. 233-257, Publication No. (NIH) 95-1468; Lowe L P, Liu K., Greenland P., Metzger B. E., Dyer A. R., Stamler J., 1997, Diabetes Care, 20:163; Wei M., Gaskill S. P., Haffner S. M., Stern M. P., 1998, Diabetes Care, 21:1167; Wei M., Gibbons L. W., Mitchell T. L., Kampert J. B., Blair S. N., 1998, CVD Prev. 1:123.

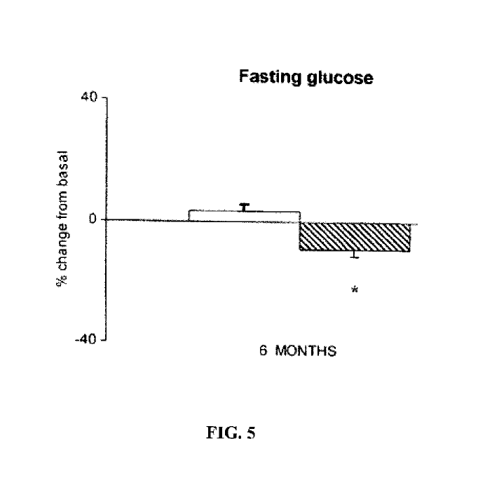

Referring now to FIG. 5, after six months of treatment, participants receiving genistein in the aforementioned study showed a decrease in levels of fasting glucose on the order of 8.7±2.3% (P<0.001) as compared to a placebo, wherein fasting glucose levels were elevated 3.2±2.3%. This decrease in glucose concentrations may decrease the risk of various chronic diseases, including diabetes, cardiovascular disease and/or the like.

Thus, genistein, according to the present invention, may be utilized to normalize fasting glucose levels. It has been determined that if blood glucose levels become too low, brain and/or heart dysfunctions may also result. See Clutter W E, Cryer P. E., 1990. Hypoglycemia, Stein J H, ed. Internal Medicine, Boston, Mass., Little Brown & Co, pgs. 2267-2272. Therefore, providing genistein to moderate fasting glucose levels may also prevent and/or reduce the risk of brain and/or heart abnormalities, including cardiovascular dysfunction, and/or cardiovascular disease and/or the like.

In another representative embodiment of the present invention a cardiovascular risk marker according to the present invention comprises fasting insulin. Insulin is a small protein that is produced in the pancreas and is secreted in response to elevated concentrations of glucose in the blood. Insulin facilitates carriage of glucose to cells. In healthy individuals, there is a surge of insulin production in response to elevated levels of glucose, but thereafter insulin levels should decrease. An elevated baseline insulin level has been known to indicate an increased risk of cardiovascular disease. See El-Atat F., Aneja A., Mcfarlane S., Sowers J., 2003. Endocrinol Metab Clin North Am., 32:823; Hall J. E., Crook E. D., Jones D. W., Wofford M. R., Dubbert P. M., 2002, Am J Med Sci, 324:127: Sowers J. R., Epstein M., Frohlich E. D., 2001, Hypertension 37:1053; Grunfeld B., Balzareti M., Romo M., Gimenez M., Gutman R. 1994, Hypertension, 23 [Suppl 1]:112; Steinberg H. O., Chaker H., Leaming R., Johnson A., Brechtel G., Baron A. D., 1996, J Clin Invest. 97:2601.

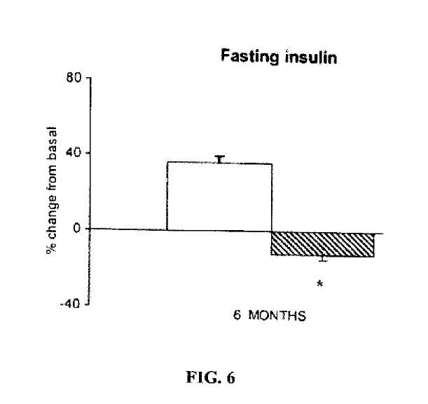

Referring now to FIG. 6, by comparison with placebo, genistein treatment significantly decreased fasting insulin levels. Specifically, genistein decreased fasting insulin levels by −12±3.33% (P>0.001), while placebo actually elevated levels by 36±3.23% (P<0.005). Through reduction in fasting insulin levels, genistein may reduce the instances of cardiovascular disease, reduce cardiovascular risk, and/or the like.

In another representative embodiment, genistein, according to various aspects of the present invention, may also be employed to lower fasting insulin levels, and thereby lower the risk and/or prevent Alzheimer's disease.

In one representative embodiment of the present invention, a cardiovascular risk marker according to the present invention may comprise insulin resistance. The production of higher than normal insulin levels to adequately absorb glucose is a condition known as insulin resistance. In individuals with this condition, normal levels do not trigger the signal for glucose absorption by cells, and thus a higher production is needed. A fasting serum insulin level of greater than the upper limit of normal for the assay used (approximately 60 pmol/L) is considered evidence of insulin resistance. There are several causes of insulin resistance, including abnormally sedentary lifestyle, haemochromatosis, hypercortisolism, and drug effects (including hormone replacement therapy). Insulin resistance has been linked to increased risk of cardiovascular disease as well as Alzheimer's disease.

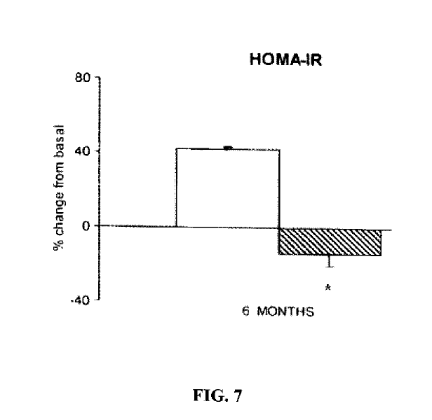

Referring now to FIG. 7, by comparison with placebo, genistein treatment significantly decreased insulin resistance levels. Specifically, genistein decreased insulin resistance levels by −14±5.8% (P>0.001), while placebo actually elevated levels by 42±0.6% (P<0.005). As discussed supra, elevated insulin levels are known to indicate an increased risk of cardiovascular disease. Through reduction of insulin resistance levels, genistein may reduce the instances of cardiovascular disease, reduce cardiovascular risk, and/or the like. In another representative embodiment, genistein, according to various aspects of the present invention, may also be employed to lower insulin resistance levels, and thereby lower the risk and/or to prevent Alzheimers disease.

In another representative embodiment in accordance with the present invention, a cardiovascular risk marker according to the present invention may comprise osteoprotegerin. Also known as an oesteoclastogenesis inhibitory factor, osteoprotegerin inhibits the differentiation of macrophages into osteoclasts as well as regulates the resorption of osteoclasts in vitro and in vivo. Osteoblasts produce and secrete osteoprotegerin to serve as a decoy receptor which can block RANKL/RANK interactions. In osteopenic and osteoporotic states, RANKL expression increases and expression of osteoprotegerin decreases. Thus, while high levels of osteoprotegerin are associated with good bone quality, high levels also tend to increase risk of cardiovascular disease and cardiovascular mortality. See Jono S., Ikari Y., Shioi A., Mori K., Miki T., Hara K., Nishizawa Y., 2002, Circulation 106:1192; Schoppet M., Sattler A. M., Schaefer J. R., Herzum M., Maisch B., Hofbauer L. C., 2003; J Clin Endocrinol Metab. 88:1024; Browner W. S., Lui L. Y., Cummings S. R., 2001, “Associations of Serum Osteoprotegerin Levels with Diabetes, Stroke, Bone Density, Fractures, and Mortality in Elderly Women”, J Clin Endocrinol Metab 86-631. Accordingly, genistein may be employed to reduce and/or normalize osteoprotegerin levels in order to reduce the risk of cardiovascular disease and/or aid in the prevention of cardiovascular disease, decrease the risk of coronary artery disease, reduce inhibition of vessel calcification and/or reduce endothelial aptosis and/or the like.

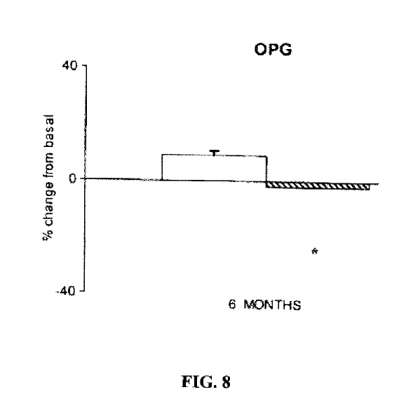

Referring now to FIG. 8, by comparison with placebo, genistein treatment decreased osteoproterin levels. Specifically, genistein decreased osteoproterin levels by −2±0.3% (P>0.001), while placebo actually elevated levels by 9±1.5% (P<0.005). Through reduction and/or stabilization of osteoproterin levels, genistein may reduce instances of cardiovascular disease, reduce cardiovascular risk, and/or the like.

In another representative embodiment of the present invention, a representative cardiovascular risk marker according to the present invention may comprise fibrinogen. As the principal protein responsible for blood clotting in mammals, fibrinogen is the primary factor which controls viscosity of whole blood and plasma in the cardiovascular system. See Drouet L., 1996, Cerebrovasc Dis, 6:2; Kannel W. B., 1997, Drugs, 54 (suppl 3):32; Harley S. L., Powell J. T., 1999, Biochem J. 341:739. Additionally, fibrinogen functions to regulate leukocyte-endothelial cell interactions. Higher levels of fibrinogen tend to lead to thrombosis (i.e., a clot inside a blood vessel, obstructing flow of blood through the circulatory system). Additionally, atherosclerosis, coronary heart disease, peripheral vascular disease and carotid stenosis and/or the like have been linked to elevated levels of fibrinogen. See Drouet L., 1996, Cerebrovasc Dis, 6:2; Kannel W. B., 1997, Drugs, 54 (suppi 3):32; Ernst E., Resch K. L., 1993, Ann Intern Med, 118:956; Maresca G., Di Blasio A., Marchioli R., Di Minno G., 1999, Arteriosder Thromb Vasc Biol, 19-1368. Due to fibrinogens direct association with the vascular system, fibrinogen levels have been found to be positively correlated with adverse vascular events. As such, employing genistein to reduce fibrinogen levels may reduce the risk and/or prevent cardiovascular disease, reduce and/or prevent atherosclerosis, coronary heart disease, peripheral vascular disease, mycoardial infartion and carotid stenosis.

As illustrated in Tables 2 and 3, by comparison with placebo, genistein treatment decreased fibrinogin levels. Specifically, genistein decreased fibrinogen levels from 3.6±0.12 g/L to 3.18±0.12 g/L, corresponding to approximately a 11.7% decrease (not taking uncertainties into account), while the placebo actually elevated levels from 3.7±0.05 g/L to 3.83±0.04 g/L. corresponding to approximately a 3.5% increase (not taking uncertainties into account) (P<0.05).

Through reduction and/or stabilization of osteoproterin levels, genistein may reduce the instances of cardiovascular disease, reduce cardiovascular risk, and/or the like.

In another representative embodiment of the present invention, a cardiovascular risk marker according to the present invention may comprise sex hormone-binding globulin. As a glycoprotein, sex hormone-binding globulin is responsible for binding to sex hormones, specifically testosterone and estradiol. By binding to sex hormones, sex hormone-binding globulin prevents the hormone from being active. Most sex hormones in the body are bound by sex hormone-binding globulin. When unbound, sex hormones are free to enter a cell and activate its receptor. Sex-hormone binding globulin appears to be decreased by high levels of insulin, and increased by high levels of estrogen. It has been shown that lower sex hormone-binding globulin levels are associated with higher insulin resistance and coronary disease in women. Thus, by increasing sex hormone binding globulin levels, a reduction in cardiovascular risk may result.

In one representative embodiment of the present invention, genistein is administered to increase sex hormone-binding globulin levels to reduce the risk of cardiovascular disease and/or the like. As illustrated by Tables 2 and 3, sex hormone binding globulin levels with administration of genistein decreased from 71±4.2 nmol/L to 63±3.83 nmol/L, corresponding to approximately a 11.3% decrease (not taking uncertainties into account), whereas serum sex hormone binding globulin levels with administration of placebo decreased from 75±2.92 nmol/L to 53±2.92 nmol/L, corresponding to approximately a 29.3% decrease (not taking uncertainties into account) (P<0.05).

In the foregoing specification, the invention has been described with reference to specific exemplary embodiments, however, it will be appreciated that various modifications and changes may be made without departing from the scope of the present invention as set forth in the claims below. The specification and figures are to be regarded in an illustrative manner, rather than a restrictive one and all such modifications are intended to be included within the scope of the present invention. Accordingly, the scope of the invention should be determined by the claims appended hereto and their legal equivalents rather than by merely the examples described above. For example, the steps recited in any method or process claims may be executed in any order and are not limited to the specific order presented in the claims.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of any or all the claims.

As used herein, the terms “comprising”, “having”, “including” or any contextual variant thereof, are intended to reference a non-exclusive inclusion, such that a process, method or composition that comprises a list of elements does not include only those elements recited, but may also include other elements not expressly listed or inherent to such process, method or composition. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

We claim:

1. A composition for improving cardiovascular function in mammals, said composition comprising substantially pure genistein.

2. The composition of claim 1, wherein genistein comprises at least one of: approximately greater than 98% pure genistein; genistein at least substantially isolated from a single plant; genistein at least substantially isolated from Glycine max; an aglycone form of the glucoside isoflavone molecule; and 4′,5,7-Trihydroxyisoflavone 5,7-Dihydroxy-3-(4-hydroxyphenyl)-4H-1-benzopyran-4-one.

3. The composition of claim 1, wherein said composition at least one of: increases plasma genistein levels without significantly affecting estradiol levels; at least one of reduces and normalizes fasting glucose levels; at least one of reduces and normalizes fasting insulin levels; at least one of reduces and normalizes insulin resistance levels; at least one of reduces and normalizes osteoprotegerin levels; reduces sex hormone-binding globulin levels, reduces fibrinogen levels; and is substantially neutral to inflammatory markers, said inflammatory markers comprising at least one of a C-reactive protein and homocysteine.

4. The composition of claim 3, wherein said composition at least one of reduces and normalizes osteoprotegerin levels, and wherein said composition at least one of: leads to a decrease in risk of coronary artery disease, reduces inhibition of vessel calcification, and reduces endothelial aptosis.

5. The composition of claim 3, wherein said composition reduces fibrinogen levels, and wherein said composition reduces risk of at least one of: atherosclerosis, coronary heart disease, peripheral vascular disease, myocardial infarction, and carotid stenosis.

6. The composition of claim 3, wherein said composition is substantially neutral to the inflammatory marker C-reactive protein, and wherein said composition reduces risk of cardiovascular disease.

7. The composition of claim 3, wherein said composition is substantially neutral to the inflammatory marker homocysteine, and wherein said composition reduces the risk of at least one of: neural tube defects, Alzheimer's disease, schizophrenia, acute renal disease, osteoporosis, and Type I diabetes.

8. The composition of claim 1 wherein said composition at least one of: substantially maintains the level of at least one inflammatory marker, and reduces the level of at least one cardiovascular risk marker for a disease.

9. The composition of claim 8, said disease comprising at least one of cardiovascular disease, diabetes osteopenia, osteoporosis, Alzheimer's disease, and dementia.

10. The composition of claim 1, further comprising at least one of: vitamin D, zinc and calcium.

11. The composition of claim 1, wherein said composition is administered at least one of orally; with a weight of 54 mg/d; in a bolus; in a metered fashion; in a time-release fashion; and daily.

12. The composition of claim 1, wherein said composition at least one of: reduces negative side effect of hormone replacement therapy; enhances dilator response to acetychioline of atheroscelerotic arteries; reduces risk of at least one of coronary heart disease, venous thrombolism, and metabolic hepatic activation; improves endothelial dependent vasodilation; comprises at least one of an anti-neoplastic effect and an anti-mutagenic effect, and at least partially inhibits at least one of: LDL oxidation, endothelial cell proliferation, and angiogenesis.

13. A method for improving cardiovascular function, said method comprising the step of orally administering a composition of substantially pure genistein.

14. The method of claim 13, wherein said genistein is administered at least one of: orally; with a weight of 54 mg/d; in a bolus; in a metered fashion; in a time-release fashion; and daily.

15. The method of claim 13, wherein genistein comprises at least one of: approximately greater than 98% pure genistein; genistein at least substantially isolated from a single plant; genistein derived from several genistein containing plants, genistein at least substantially isolated from Glycine max; an aglycone form of the glucoside isoflavone molecule; and 4′, 5,7-Trihydroxyisoflavone 5,7-Dihydroxy-3-(4-hydroxyphenyl)-4H-1-benzopyran-4-one.

16. The method of claim 13, wherein said composition at least one of: increases plasma genistein levels without significantly affecting estradiol levels, at least one of reduces and normalizes fasting glucose levels; at least one of reduces and normalizes fasting insulin levels; at least one of reduces and normalizes insulin resistance levels; at least one of reduces and normalizes osteoprotegerin levels; reduces sex hormone-binding globulin levels; reduces fibrinogen levels; is substantially neutral to inflammatory markers, said inflammatory markers comprising at least one of a C-reactive protein and homocysteine.

17. The method of claim 16, wherein said method at least one of reduces and normalizes osteoprotegerin levels, and wherein said composition at least one of: leads to a decrease in risk of coronary artery disease, reduces inhibition of vessel calcification, and reduces endothelial aptosis.

18. The method of claim 16, wherein said method reduces fibrinogen levels, and wherein said composition reduces a risk of at least one of: atherosclerosis, coronary heart disease, peripheral vascular disease, myocardial infarction, and carotid stenosis.

19. The method of claim 16, wherein said method is substantially neutral to the inflammatory marker C-reactive protein, and wherein said composition reduces risk of cardiovascular disease.

20. The method of claim 16, wherein said method is substantially neutral to the inflammatory marker homocysteine, and wherein said method reduces the risk of at least one of: neural tube defects, Alzheimers disease, schizophrenia, acute renal disease, osteoporosis, and Type I diabetes.

21. The method of claim 13, said method further comprising the step of administering at least one of: vitamin D, zinc and calcium.

22. The method of claim 13, wherein said method at least one of: reduces negative side effect of hormone replacement therapy; enhances dilator response to acetychioline of atheroscelerotic arteries; reduces risk of at least one of coronary heart disease, venous thrombolism; and metabolic hepatic activation; improves endothelial dependent vasodilation; comprises at least one of an anti-neoplastic effect and an anti-mutagenic effect; and at least partially inhibits at least one of: LDL oxidation, endothelial cell proliferation, and angiogenesis.

23. A composition for at least one of: substantially maintaining the level of at least one inflammatory marker, and at least one of normalizing and reducing the level of at least one cardiovascular risk marker for a disease; said composition comprising substantially pure genistein.

24. The composition of claim 23, wherein said genistein comprises at least one of: approximately greater than 98% pure genistein; genistein at least substantially isolated from a single plant; genistein at least substantially isolated from Glycine max; an aglycone form of the glucoside isoflavone molecule; and 4,5,7-Trihydroxyisoflavone 5,7-Dihydroxy-3-(4-hydroxyphenyl)-4H-1-benzopyran-4-one.

25. The composition of claim 23, wherein said disease comprises at least one of: cardiovascular disease, diabetes, osteopenia, osteoporosis, Alzheimers disease, and dementia.

26. The composition of claim 23, further comprising at least one of: vitamin D, zinc, and calcium.

27. The composition of claim 23, wherein said composition is administered at least one of: orally; with a weight of 54 mg/d; in a bolus; in a metered fashion; in a time-release fashion, and daily.

28. The composition of claim 23, wherein said inflammatory marker comprises at least one of: C-reactive protein and homocysteine.

29. The composition of claim 28, wherein said composition is substantially neutral to the inflammatory marker homocysteine, and wherein said composition reduces the risk of at least one of: neural tube defects, Alzheimer's disease, schizophrenia, acute renal disease, osteoporosis, and Type I diabetes.

30. The composition of claim 28, wherein said composition is substantially neutral to the inflammatory marker C-reactive protein, and wherein said composition reduces risk of cardiovascular disease.

31. The composition of claim 23, wherein said cardiovascular risk marker comprises at least one of: plasma genistein, fasting glucose, fasting insulin, osteoprotegerin, sex hormone-binding globulin, and fibrinogen.

32. The composition of claim 31, wherein said composition at least one of normalizes and reduces osteoprotegerin levels, and wherein said composition at least one of: leads to a decreased risk of coronary artery disease, reduction of inhibition of vessel calcification, and reduction of endothelial aptosis.

33. The composition of claim 31, wherein said composition reduces fibrinogen levels, and wherein said composition reduces a risk of at least one of: atherosclerosis, coronary heart disease, peripheral vascular disease, myocardial infarction, and carotid stenosis.

34. The composition of claim 23, wherein said composition at least one of: reduces negative side effect of hormone replacement therapy; enhances dilator response to acetychloline of atheroscelerotic arteries; reduces risk of at least one of coronary heart disease, venous thrombolism, and metabolic hepatic activation; improves endothelial dependent vasodilation; comprises at least one of an anti-neoplastic effect and an anti-mutagenic effect; and at least partially inhibits at least one of: LDL oxidation, endothelial cell proliferation, and angiogenesis.

35. A method for at least one of: substantially maintaining the level of at least one inflammatory marker, and at least one of normalizing and reducing the level of at least one cardiovascular risk marker for a disease; said method comprising the step of providing a composition comprising substantially pure genistein.

36. The method of claim 35, wherein said genistein comprises at least one of: approximately greater than 98% pure genistein; genistein at least substantially isolated from a single plant; genistein at least substantially isolated from Glycine max; an aglycone form of the glucoside isoflavone molecule; and 4′,5,7-Trihydroxyisoflavone 5,7-Dihydroxy-3-(4-hydroxyphenyl)-4H-1-benzopyran-4-one.

37. The method of claim 35, wherein said disease comprises at least one of: cardiovascular disease, diabetes, osteopenia, osteoporosis, Alzheimer's disease, and dementia.

38. The method of claim 35, said composition further comprising at least one of: vitamin D, zinc, and calcium.

39. The method of claim 35, wherein said composition is administered at least one of: orally, with a weight of 54 mg/d; in a bolus; in a metered fashion; in a time-release fashion; and daily.

40. The method of claim 35, wherein said inflammatory marker comprises at least one of: C-reactive protein and homocysteine.

41. The method of claim 40, wherein said composition is substantially neutral to the inflammatory marker homocysteine, and wherein said composition reduces the risk of at least one of: neural tube defects, Alzheimer's disease, schizophrenia, acute renal disease, osteoporosis, and Type I diabetes.

42. The method of claim 40, wherein said composition is substantially neutral to the inflammatory marker C-reactive protein, and wherein said composition reduces risk of cardiovascular disease.

43. The method of claim 35, wherein said cardiovascular risk marker comprises at least one of: plasma genistein, fasting glucose, fasting insulin, osteoprotegerin, sex hormone-binding globulin, and fibrinogen.

44. The method of claim 43, wherein said composition at least one of normalizes and reduces osteoprotegerin levels, and wherein said composition at least one of: leads to a decreased risk of coronary artery disease, reduction of inhibition of vessel calcification, and reduction of endothelial aptosis.

45. The method of claim 43, wherein said composition reduces fibrinogen levels, and wherein said composition reduces a risk of at least one of: atherosclerosis, coronary heart disease, peripheral vascular disease, myocardial infarction, and carotid stenosis.

46. The method of claim 35, wherein said composition at least one of: reduces negative side effect of hormone replacement therapy; enhances dilator response to acetychloline of atheroscelerotic arteries; reduces risk of at least one of coronary heart disease, venous thrombolism, and metabolic hepatic activation; improves endothelial dependent vasodilation; comprises at least one of an anti-neoplastic effect and an anti-mutagenic effect, and at least partially inhibits at least one of: LDL oxidation, endothelial cell proliferation, and angiogenesis.

47. A composition for reducing cardiovascular risk factors, said composition comprising substantially pure genistein, wherein said composition is administered to at least one: increase plasma genistein levels without significantly affecting estradiol levels; at least one of reduce and normalize fasting glucose levels; at least one of reduce and normalize fasting insulin levels; at least one of reduce and normalizes insulin resistance levels; at least one of reduce and normalize osteoprotegerin levels; reduce sex hormone-binding globulin levels; reduce fibrinogen levels; is substantially neutral to inflammatory markers, said inflammatory markers comprising at least one of a C-reactive protein and homocysteine.

48. The composition of claim 47, wherein genistein comprises at least one of: approximately greater than 98% pure genistein; genistein at least substantially isolated from a single plant; genistein at least substantially isolated from Glycine max; an aglycone form of the glucoside isoflavone molecule; and 4′,5,7-Trihydroxyisoflavone 5,7-Dihydroxy-3-(4-hydroxyphenyl)-4H-1-benzopyran-4-one.

49. The composition of claim 47, wherein said composition at least one of reduces and normalizes osteoprotegerin levels, and wherein said composition at least one of, leads to a decrease in risk of coronary artery disease, reduces inhibition of vessel calcification, and reduces endothelial aptosis.

50. The composition of claim 47, wherein said composition reduces fibrinogen levels, and wherein said composition reduces a risk of at least one of atherosclerosis, coronary heart disease, peripheral vascular disease, myocardial infarction, and carotid stenosis.

51. The composition of claim 47, wherein said composition is substantially neutral to the inflammatory marker C-reactive protein, and wherein said composition reduces risk of cardiovascular disease.

52. The composition of claim 47, wherein said composition is substantially neutral to the inflammatory marker homocysteine, and wherein said composition reduces the risk of at least one of: neural tube defects, Alzheimer's disease, schizophrenia, acute renal disease, osteoporosis, and Type I diabetes.

53. The composition of claim 47, wherein said disease comprises at least one of: cardiovascular disease, diabetes, osteopenia, osteoporosis, Alzheimers disease, and dementia.

54. The composition of claim 47, further comprising at least one of: vitamin D, zinc and calcium.

55. The composition of claim 47, wherein said composition is administered at least one of: orally; with a weight of 54 mg/d; in a bolus; in a metered fashion; in a time-release fashion; and daily.

56. The composition of claim 47, wherein said composition at least one of: reduces negative side effect of hormone replacement therapy; enhances dilator response to acetychloline of atheroscelerotic arteries; reduces risk of at least one of coronary heart disease, venous thrombolism, and metabolic hepatic activation, improves endothelial dependent vasodilation; comprises at least one of an anti-neoplastic effect and an anti-mutagenic effect; and at least partially inhibits at least one oft LDL oxidation, endothelial cell proliferation, and angiogenesis.

57. The composition of claim 47, wherein said composition is administered to lower fasting glucose levels approximately 8.7±2.3%.

58. The composition of claim 47, wherein said composition is administered to lower fasting insulin levels approximately 12±3.33%.

59. The composition of claim 47, wherein said composition is administered to lower insulin resistance levels approximately 14±5.8%.

60. The composition of claim 47, wherein said composition is administered to increase plasma genistein levels approximately 60%.

61. The composition of claim 47, wherein said composition is administered to lower osteoproterin levels approximately 2±0.3%.

62. The composition of claim 47, wherein said composition is administered to lower fibrinogen levels by approximately 11.7%.

63. The composition of claim 47, wherein said composition is administered to lower sex hormone-binding globulin by approximately 11.3%.
